SEQUENCE LISTING (12 sequences)

SEQ ID NO: 1 - protein, 495 aa, Chelatococcus asaccharovorans
MSAHVADATRIGRASGAKAPEFQELYDFDAAALTLTSAVFPYDSKIHRAHVVMLAEQAILTRDEASRILHGLAAADERASIDASLRTYLPYEAALKGEIGPVAGKMHIGRSRNDLANAGKRMFLRDQLLRTIEAVIGLRHAVVDKAAGHLDTVMVVYTQRKEAQPITLGHYLMAISENLGKNLDRFRELYPRVNQCPLGAAATAGTGWPLDRDRTADLLGFDGLVVNSIEGVAGWDHVAEFAFDNAIFLSGLSRLASEIQLWSSDEYQMAELDASFAGTSSIMPQKKNPDSLERSRKAAFAAMGPLVGILTSLNGIEYQYSAARVELEPRSIDAMIAATHAMTGVVRTLHPNKEQMLRYAAENYATMTDLTDMLVRRVGIDYREAHEIVARVVMAAIDQGIKANRIGLDLVQQAAIAQTGQPIAVSADDIADALDPVQNVARRKGKGMPAPESVQTAIDDARLELRTDHAWLDSARQRLAAADAALATAVEAIRA

SEQ ID NO: 2 - DNA, 1497 bp, synthetic construct
  source 1..1497: mol_type = other DNA
catatgagtgcccatgttgcagatgccacccgtattggccgtgccagtggcgccaaagccccggagttccaggaactgtatgacttcgatgccgccgccctgaccctgaccagtgccgtgttcccgtatgatagtaaaattcatcgcgcccatgtggttatgctggccgaacaggccattctgacccgtgatgaagccagtcgtattctgcatggtctggccgccgcagatgaacgtgccagtattgatgcaagcctgcgtacctatctgccgtatgaagccgccctgaaaggcgaaattggcccggtggcaggtaaaatgcatattggccgtagtcgcaatgatctggcaaatgcaggcaaacgtatgttcctgcgtgatcagctgctgcgcaccattgaagcagttattggtctgcgccatgcagttgttgataaagccgcaggtcatctggataccgtgatggtggtgtatacccagcgtaaagaagcacagccgattaccctgggccattatctgatggccattagcgaaaatctgggtaaaaatctggatcgcttccgtgaactgtatccgcgtgtgaatcagtgtccgctgggcgcagccgcaaccgcaggtacaggttggccgctggatcgtgatcgtaccgcagacttactgggcttcgatggcctggttgtgaatagcattgaaggcgttgcaggctgggatcatgttgccgagttcgccttcgataatgccatcttcctgagcggtctgagtcgtctggccagcgaaattcagctgtggagtagcgatgaatatcagatggcagaactggatgcatcattcgcaggcaccagtagtattatgccgcagaaaaaaaatccggatagcctggaacgtagtcgtaaagcagcattcgcagcaatgggtccgctggttggcattctgaccagcctgaatggcattgaatatcagtatagtgcagcacgtgttgaactggaaccgcgtagtattgatgccatgattgccgccacccatgcaatgaccggcgttgttcgcaccctgcatccgaataaagaacagatgctgcgttatgcagcagaaaattatgcaaccatgaccgatctgaccgatatgctggttcgtcgcgttggcattgattatcgtgaagcacatgaaattgttgcccgcgtggtgatggccgcaattgatcagggtattaaagccaatcgtattggtctggatctggttcagcaggccgcaattgcacagaccggtcagccgattgccgtgagtgcagatgatattgcagatgccctggaccctgttcagaatgttgcccgccgtaaaggcaaaggtatgccggcaccggaaagcgttcagaccgccattgatgatgcacgtctggaactgcgcaccgatcatgcatggctggatagtgcccgccagcgtctggccgccgctgatgcagcactggcaaccgcagtggaagccattcgcgcctaaggatcc

SEQ ID NO: 3 - protein, 609 aa, Bradyrhizobium sp. SK17
MRSMIADRHPVSHHIDSAGGVCPPCEGHGADRAIWTDSRIAIVMQPCIKAIAPAGRSQHDPAEHRPSVSAPRAVRNPIRRASGPMTLLTARAVLTASCSLVLAAALATASTRAQQNPALNDGTRIGRSAVPKAPEFQELYDFDAAAKTLGDATFPYEVRVHRAHVVMLAEKGIVTRSDAATILRGLDTVDARAADDASLRTYLPYEAALIKSIGPVAGRMHTGRSRNDLANTVNRMFYRDQLNRTVEALIALRSAVVAKAADNLDTVMVVYTHRKEAQPITLGHYLMAISESLGKSIDRYEQLYARVNQSPLGAAASAGTSWPLDRERTAALLGFDGLVIDTIEGTAGWDHIAEFASDNAIYLSGLSRLASEIQLWSTDEYRSAELDPAFAGTSSIMPQKKNPDSLERTRQIAANSVGAVTSVLTSLNAAEYQHSVTRVPLEPRSLDAMIAATHAMTGVVRTLQPNKEQMLRYAAQNFSTMTDLADTIVRESGIDFREAHEIIARVVEAAINGGKTADQIDVAMIETAAQAQLGRTIQISAAAVRDALDPVRSVKLRNGIGGPAASSVAAMIKVSQAEISDEQSRLAARRQKIAAASAALAQAVAAAEH

SEQ ID NO: 4 - DNA, 1839 bp, synthetic construct
  source 1..1839: mol_type = other DNA
catatgcgcagcatgattgcagatcgccatccggttagtcatcatattgatagcgcaggtggcgtgtgtccgccgtgcgaaggtcatggcgcagatcgcgccatctggaccgatagtcgtattgcaattgttatgcagccgtgtattaaagcaattgcaccggccggccgcagtcagcatgatccggctgaacatcgcccgagtgttagcgccccgcgtgccgttcgtaatccgattcgccgtgccagtggcccgatgaccctgctgaccgcacgcgcagtgctgaccgcaagttgcagcctggtgctggccgcagccctggcaaccgcaagtacccgcgcccagcagaatccggcactgaatgatggtacccgtattggtcgtagtgcagttccgaaagccccggagttccaggaactgtatgacttcgatgccgccgccaaaaccttaggcgatgcaaccttcccgtatgaagttcgtgtgcatcgcgcccatgtggtgatgctggcagaaaaaggtattgttacccgtagtgatgccgcaaccattctgcgtggcctggataccgtggatgcccgtgccgccgatgatgcaagcctgcgcacctatctgccgtatgaagccgcactgattaaaagcattggtccggttgcaggtcgcatgcataccggccgtagccgcaatgatctggcaaataccgttaatcgcatgttctatcgtgatcagctgaatcgtaccgttgaagcactgattgcactgcgtagtgcagtggttgccaaagcagcagataatctggataccgttatggtggtgtatacccatcgtaaagaagcacagccgattaccctgggtcattatctgatggcaattagtgaaagcctgggcaaaagcattgatcgttatgaacagctgtatgcacgtgtgaatcagagtccgctgggtgccgccgccagcgcaggtaccagttggcctctggatcgcgaacgtaccgcagccctgctgggcttcgatggtctggttattgataccattgaaggtaccgccggctgggatcatattgccgagttcgccagcgataatgcaatctatctgagtggtctgagccgcctggccagcgaaattcagctgtggagcaccgatgaatatcgtagtgccgaactggaccctgcattcgcaggtaccagcagcattatgccgcagaaaaaaaatccggatagcctggaacgtacccgtcagattgcagcaaatagcgttggtgccgttaccagcgttctgaccagcctgaatgcagccgaatatcagcatagtgttacccgtgtgccgctggaaccgcgcagcctggatgccatgattgcagccacccatgccatgaccggcgttgtgcgtaccctgcaaccgaataaagaacagatgctgcgttatgccgcccagaacttcagcaccatgaccgatctggccgataccattgttcgtgaaagcggcattgacttccgtgaagcccatgaaattattgcccgcgttgtggaagccgcaattaatggtggtaaaaccgccgatcagattgatgttgcaatgattgaaaccgcagcccaggcccagctgggtcgcacaattcagattagcgcagccgccgtgcgcgatgcactggaccctgtgcgcagtgttaaactgcgcaatggtattggtggtccggcagccagcagtgtggcagccatgattaaagttagtcaggccgaaattagcgatgaacagagccgcctggcggcccgccgtcaaaaaattgccgcagcaagtgccgcactggcccaggcagttgcagccgctgaacattaaggatcc

SEQ ID NO: 5 - protein, 525 aa, Bradyrhizobium elkanii
MTLLTARAVLTASCSLVLAAALATASTRAQQNPALNDGTRIGRSAVPKAPEFQELYDFDAAAKTLGDATFPYEVRVHRAHVVMLAEKGIVTRSDAATILRGLDTVDARAADDASLRTYLPYEAALIKTIGPVAGRMHTGRSRNDLANTVNRMFYRDQLNRTVEALIALRSAVVAKAADNLDTVMVVYTHRKEAQPITLGHYLMAISESLGKSIDRYEQLYARVNQSPLGAAASAGTSWPLDRERTAALLGFDGLVIDTIEGTAGWDHIAEFASDNAIYLSGLSRLASEIQLWSTDEYRSAELDPAFAGTSSIMPQKKNPDSLERTRQIAANSVGAVTSVLTSLNAAEYQHSVTRVPLEPRSLDAMIAATHAMTGVVRTLQPNKEQMLRYAAQNFSTMTDLADTIVRESGIDFREAHEIIARVVEAAINGGKTADQIDVAMIETAAQAQLGRTIQISAAAVRDALDPVRSVKLRNGIGGPAASSVAAMIKVSQAEISDEQSRLAARRQKIAAASAALAQAVAAAEH

SEQ ID NO: 6 - DNA, 1587 bp, synthetic construct
  source 1..1587: mol_type = other DNA
catatgaccctgctgaccgcacgtgcagttctgaccgcaagctgtagcctggttctggccgcagccctggcaaccgcaagcacccgtgcccagcagaatccggcactgaatgatggtacccgtattggccgtagtgcagtgccgaaagccccggagttccaggaactgtatgacttcgatgcagccgcaaaaaccttaggtgatgcaaccttcccgtatgaagttcgcgtgcatcgcgcacatgtggtgatgctggccgaaaaaggtattgttacccgcagtgatgcagcaaccattctgcgtggtctggataccgttgatgcacgcgccgcagatgatgcaagcctgcgcacctatctgccgtatgaagcagcactgattaaaaccattggtccggtggccggtcgcatgcataccggccgtagtcgtaatgatctggcaaataccgtgaatcgtatgttctatcgcgatcagctgaatcgtaccgtggaagccctgattgcactgcgcagcgccgtggttgcaaaagcagcagataatctggataccgtgatggttgtgtatacccatcgcaaagaagcacagccgattaccctgggccattatctgatggcaattagcgaaagcctgggcaaaagtattgatcgctatgaacagctgtatgcccgtgttaatcagagcccgctgggtgcagcagcaagcgccggtaccagctggccgctggatcgcgaacgtaccgcagccctgctgggcttcgatggcctggttattgataccattgaaggtaccgccggttgggatcatattgcagagttcgccagtgataatgcaatctatctgagcggtctgagtcgtctggcaagtgaaattcagctgtggagtaccgatgaatatcgtagcgccgaactggaccctgccttcgccggcaccagtagcattatgccgcagaaaaaaaatccggatagtctggaacgcacccgtcagattgcagccaatagcgttggtgccgttaccagcgttctgaccagcctgaatgcagccgaatatcagcatagcgtgacccgtgtgccgctggaaccgcgtagcctggatgcaatgattgcagccacccatgcaatgaccggcgtggttcgcaccctgcaaccgaataaagaacagatgctgcgctatgccgcccagaacttcagcaccatgaccgatctggcagataccattgtgcgcgaaagcggtattgacttccgtgaagcacatgaaattattgcacgtgtggtggaagccgcaattaatggcggcaaaaccgcagatcagattgatgttgcaatgattgaaaccgcagcacaggcccagctgggccgtaccattcagattagtgcagccgccgttcgcgatgccctggaccctgttcgcagcgttaaactgcgtaatggcattggtggcccggccgcaagtagtgtggcagcaatgattaaagttagccaggcagaaattagtgatgaacagagccgtctggccgcacgccgccagaaaattgcagccgcaagcgcagcactggcacaggcagtggcagccgccgaacattaaggatcc

SEQ ID NO: 7 - protein, 499 aa, Caballeronia pedi
MSNTQGVKDATRIGRSSVAKAPEFQELYDFDAAAKTLVDATFPYESDIHHAHVVMLAECDIIKRDEAAQILRGLEAVDAQAKENPSLRTYLPYEAALKKAVGAVGGKMHLGRSRNDLANAVKRMFLRDQLNRVIEAVIALRASVVAKAADHLDTVMVVYTQRKEAQPITLGHYLMAYAESLGKSLDRYEQLYARINQCPLGAAATAGTGWPLDRERTSALLGFDSLVVNTIEGVAGWDHIAEFAADNAIYMSSLSRLASEIQLWSTDEYRSAELDPAFAGTSSIMPQKKNPDSLERTRQAAFATMGPLVAILTSLNGIEYQYSAARVGLEPRSIDGMIAATHAMTGVVRTLQPNAAQMLRYAAENFATMTDLTDMLVRKTGLDYREAHEVIARVVNAALDQGKKANVIDLDLVQDAARAQLGHTLDISATDVRDALDPVQNVARRNGIGGPAPVAVQRTIDTAQRELDEQRACYDTRMRKLADASRERARAVASMTAGS

SEQ ID NO: 8 - DNA, 1509 bp, synthetic construct
  source 1..1509: mol_type = other DNA
catatgagcaatacccagggtgttaaagatgccacccgcattggtcgcagtagcgttgccaaagccccggagttccaggaactgtatgacttcgatgcagccgcaaaaaccttagttgatgccaccttcccgtatgaaagcgatattcatcatgcccatgtggttatgctggccgaatgtgatattattaaacgcgatgaagcagcacagattctgcgtggcctggaagccgtggatgcacaggcaaaagaaaatccgagcctgcgcacctatctgccgtatgaagccgcactgaaaaaagcagttggcgccgtgggtggtaaaatgcatctgggtcgtagccgcaatgatctggccaatgcagtgaaacgcatgttcctgcgtgatcagctgaatcgtgtgattgaagcagtgattgcactgcgcgcaagtgttgttgccaaagcagccgatcatctggataccgtgatggttgtgtatacccagcgtaaagaagcccagccgattaccctgggtcattatctgatggcatacgctgaaagtctgggcaaaagcctggatcgttatgaacagctgtatgcacgtattaatcagtgcccgctgggtgccgcagccaccgcaggtacaggctggcctctggatcgtgaacgcaccagcgcactgctgggcttcgatagcctggtggttaataccattgaaggtgtggcaggctgggatcatattgccgagttcgcagcagataatgcaatctatatgagtagcctgagtcgtctggcaagcgaaattcagctgtggagcaccgatgaatatcgcagcgcagaactggaccctgccttcgccggtaccagcagtattatgccgcagaaaaaaaatccggatagcctggaacgcacccgccaggccgccttcgctaccatgggccctctggttgccattctgaccagtctgaatggcattgaatatcagtatagcgccgcacgtgttggcctggaaccgcgtagtattgatggtatgattgccgcaacccatgccatgaccggcgttgttcgtaccctgcaaccgaatgccgcacagatgctgcgctatgcagcagaaaacttcgccaccatgaccgatctgaccgatatgctggtgcgcaaaaccggtctggattatcgcgaagcccatgaagttattgcccgtgttgttaatgccgcactggatcagggtaaaaaagccaatgtgattgatctggatctggtgcaggatgccgcacgtgcacagctgggtcataccctggatattagtgcaaccgatgtgcgtgatgccctggaccctgttcagaatgtggcacgccgcaatggtattggtggtccggcaccggttgcagtgcagcgtaccattgataccgcacagcgcgaactggatgaacagcgtgcctgctatgatacccgcatgcgtaaactggcagatgcaagtcgtgaacgtgcccgcgcagttgccagtatgaccgccggcagttaaggatcc

SEQ ID NO: 9 - protein, 498 aa, Paraburkholderia sartisoli
MSDKQGVVDATRIGRSSVAKAPEFQELYEFDAAAKTLVDATFPYESQIHRAHVVMLAESGILKTQEVAEILRGLAVVDERARSDPSLRTYLPYEAALKKVVGSVGGKMHLGRSRNDLANAVKRMFLRDQANRTIEALIGLRAAVIAKAADNLDTVMVVYTQRKEAQPITLAHYLMAYSEGLGKSIERYEQMYARINHCPLGAAATAGTGWPLNRERTSGLLGFDALVVNTIEGVAGWDHIAEFAANNAIYMSSLSRLASEIQLWSTDEYRVAELDPSFAGTSSIMPQKKNPDSLERTRQAAAAAMGPFVSIVTSLNAIEYQYSAARVGLEPRSIDGIMAATHAMTGVVRTLQPNKAQMLKYASENFATMTDLTDMLVRECGLDYREAHEVIARVVNAAIDARKTANQIDLDMVQAAAHAQLGHPLDISAAAIAAALDPVQNVGRRNGIGGPAPSSVQQMIDNDRKALAEEQARLEDRFARLKHSGTDLDDAVKRIAGL

SEQ ID NO: 10 - DNA, 1506 bp, synthetic construct
  source 1..1506: mol_type = other DNA
catatgagtgataagcagggtgtggtggatgcaacccgcattggccgcagtagtgttgcaaaagcaccggagttccaggaactgtatgagttcgatgcagcagcaaaaaccttagtggatgcaacattcccgtatgaaagtcagattcatcgtgcccatgtggttatgctggcagaaagcggtattctgaaaacacaagaagtggcagaaattctgcgcggcctggcagtggttgatgaacgtgcccgcagcgatccgagcctgcgtacctatctgccgtatgaagcagccctgaaaaaagtggtgggcagcgttggcggtaaaatgcatctgggccgcagtcgtaatgatctggccaatgcagtgaaacgcatgttcctgcgtgatcaggcaaatcgcaccattgaagccctgattggtctgcgtgcagccgtgattgccaaagcagccgataatctggataccgttatggtggtgtatacccagcgtaaagaagcacagccgattaccctggcacattatctgatggcctatagcgaaggtctgggtaaaagcattgaacgctatgaacagatgtatgcacgtattaatcattgcccgctgggtgccgcagccaccgcaggtacaggctggcctctgaatcgcgaacgtaccagtggcctgctgggcttcgatgcactggttgtgaataccattgaaggtgttgcaggttgggatcatattgccgagttcgccgcaaataatgcaatctatatgagcagcctgagccgtctggcaagtgaaattcagctgtggagtaccgatgaatatcgtgttgccgaactggaccctagcttcgcaggtaccagcagtattatgccgcagaaaaaaaatccggatagtctggaacgtacccgtcaggccgccgccgctgctatgggtccgttcgtgagtattgttaccagcctgaatgccattgaatatcagtatagcgccgcccgtgtgggtctggaaccgcgtagtattgatggcattatggccgcaacccatgccatgaccggcgtggttcgcaccctgcaaccgaataaagcccagatgctgaaatatgccagcgaaaacttcgcaaccatgaccgatctgaccgatatgctggtgcgtgaatgtggcctggattatcgtgaagcccatgaagttattgcccgtgttgtgaatgccgcaattgatgcacgtaaaaccgccaatcagattgatctggatatggtgcaggcagccgcacatgcacagctgggccatccgctggatattagcgcagccgcaattgcagcagcactggaccctgtgcagaatgttggccgtcgtaatggcattggtggcccggcaccgagcagtgtgcagcagatgattgataatgatcgtaaagcactggccgaagaacaggcccgcctggaagatcgcttcgcccgcctgaaacatagtggtaccgatctggatgatgcagttaaacgtattgcaggtctgtaaggatcc

SEQ ID NO: 11 - protein, 495 aa, Brevundimonas sp. TN-3
MTPHNPDATRIGRAKRAKAPEFQELYDFEAAALTLTSAVFPYDSKIHRAHVVMLAEQDILTRDEAASILNGLAKADELAGKDAALRTYLPYEAALKREIGSVAGKMHIGRSANDLRNRVNACSCVTALRTVEAVIALREAVVTKAADHLDTVMVVYTQRKEAQPITLGHYLMAISENLGKNLARYRELHPRINQCPLGAAATAGTGWPLDRDRTAALLGFHGLVVNSIEGVAGWDHVAEFAFDNAVFLSGLSRLASEIQLWSTDEYQMAELDSAFAGTSSIMPQKKNPDSLERSRKGAFAAMGPLVAILTSLNGIEYQYSAARVELEPRSIDALIAATHAMTGVVRTLHPNKEQDACAMRQENYATMTDLTDLLVRRIGIDYREAHEIVARVVMTAIERGIKANAIGLDLVQEAAVAQTGNRIEIGAADIADALDPVQNVARRKGRGMPAPESVRAAIAEARQELDADKAWLEDRRAGLADADAALEEAVAGITT

SEQ ID NO: 12 - DNA, 1497 bp, synthetic construct
  source 1..1497: mol_type = other DNA
catatgaccccgcataatccggatgcaacccgcattggtcgtgcaaaacgcgcaaaagcaccggaatttcaggaactgtatgattttgaagccgcagcactgaccctgaccagcgcagtttttccgtatgatagcaaaattcatcgtgcacatgtggttatgctggcagaacaggatattctgacccgcgatgaagcagcaagcattctgaatggtctggcaaaagcagatgaactggccggcaaagatgccgccctgcgtacctatctgccgtatgaagcagccctgaaacgcgaaattggcagcgttgccggcaaaatgcatattggccgtagtgcaaatgatctgcgcaatcgcgtgaatgcatgcagttgtgttaccgccctgcgtacagttgaagccgttattgccctgcgtgaagcagttgttaccaaagcagccgatcatctggataccgttatggttgtttatacccagcgcaaagaagcccagccgattaccctgggtcattatctgatggcaattagtgaaaatctgggcaaaaatctggcccgctatcgcgaactgcatccgcgcattaatcagtgtccgctgggcgcagccgccaccgcaggtaccggttggcctctggatcgcgatcgtaccgcagccctgctgggttttcatggcctggtggttaatagcattgaaggcgtggcaggctgggatcatgttgcagaatttgcatttgataatgcagtttttctgagtggtctgagtcgcctggccagcgaaattcagctgtggagtaccgatgaatatcagatggcagaactggatagcgcctttgccggcaccagtagcattatgccgcagaaaaaaaatccggatagtctggaacgtagccgcaaaggtgcatttgccgccatgggtccgctggtggcaattctgaccagcctgaatggtattgaatatcagtatagcgccgcccgcgtggaactggaaccgcgtagtattgatgcactgattgccgcaacccatgccatgaccggtgtggtgcgcaccctgcatccgaataaagaacaggatgcatgtgccatgcgtcaggaaaattatgccaccatgaccgatctgaccgatctgctggttcgccgtattggtattgattatcgtgaagcccatgaaattgtggcccgcgttgttatgaccgcaattgaacgtggcattaaagccaatgccattggtctggatctggtgcaggaagccgccgttgcccagaccggtaatcgcattgaaattggtgcagcagatattgccgatgccctggaccctgtgcagaatgtggcacgccgtaaaggtcgtggtatgccggccccggaaagtgttcgtgcagccattgccgaagcccgtcaggaactggatgcagataaagcctggctggaagatcgtcgcgccggcctggcagatgcagatgcagccctggaagaagcagtggccggtattaccacctgaggatcc

(19) United States
(12) Patent Application Publication
(10) Pub. No.: US 2026/0209807 A1
(43) Pub. Date: Jul. 23, 2026

(54) NEW ETHYLENEDIAMINE-N,N'-DISUCCINIC ACID (EDDS) SYNTHASES

(71) Applicant: BASF SE, Ludwigshafen am Rhein (DE)

(72) Inventors: Marta REINOSO GARCIA, Ludwigshafen am Rhein (DE); Eva MAHLER, Ludwigshafen am Rhein (DE); David Martin MAURER, Ludwigshafen am Rhein (DE); Andrea HEROLD, Ludwigshafen am Rhein (DE); Stefan SEEMAYER, Ludwigshafen (DE); Gunther ZIMMERMANN, Ludwigshafen (DE)

(21) Appl. No.: 19/139,224

(22) PCT Filed: Dec. 6, 2023
(86) PCT No.: PCT/EP2023/084523

§ 371 Date: Jun. 13, 2025

(30) Foreign Application Priority Data
Dec. 16, 2022 (EP) 22214052.7

(51) Int. Cl. C12P 13/04; C12N 9/88; C12N 15/70; C12R 1/19

(52) U.S. Cl. CPC C12P 13/04; C12N 9/88; C12N 15/70; C12R 2001/19; C12Y 402/01002; C12Y 403/02001

(57) ABSTRACT

In the present invention new EDDS synthases are provided. More specifically, new polypeptides having EDDS synthase activity, methods of using the EDDS synthases to produce EDDS, methods of producing a composition comprising EDDS and methods of making the EDDS synthases are provided.

### FIELD OF THE INVENTION

The present invention provides new ethylenediamine-N,N′-disuccinic acid (EDDS) synthases. More specifically, new polypeptides having EDDS synthase activity, methods of using the EDDS synthases to produce EDDS, methods of producing a composition comprising EDDS and methods of making the EDDS synthase are provided.

### BACKGROUND OF THE INVENTION

Enzymes are protein-based biocatalysts widely used in a variety of different industries. They can be very specific and enantioselective catalysts and can be employed under milder reaction conditions in comparison to traditional catalysts. They, for example, can be catalytically active at lower temperatures, which can result in an overall reduction of energy consumption of production processes. Additionally, enzymes are biodegradable and therefore increasingly used in various applications as sustainable alternative to petrochemistry.

Ethylenediamine-N,N′-disuccinic acid (EDDS) is a chelating agent that can bind metal ions in a stable chelator complex. (S,S)-EDDS is readily biodegradable, has low toxicity and is therefore an attractive alternative to traditional chelating agents such as ethylenediaminetetraacetic acid (EDTA). EDTA is a synthetic chelator, currently widely used in industry, with very similar structure and properties to EDDS. However, EDTA is not readily biodegradable.

Hence, there is a need for identifying new enzymes for the sustainable production of biodegradable EDDS.

### BRIEF SUMMARY OF THE INVENTION

Therefore, the present invention is directed to an isolated, synthetic, or recombinant polypeptide having EDDS synthase activity comprising an amino acid sequence that is at least 87% identical to SEQ ID NO: 1, an amino acid sequence that is at least 80% identical to SEQ ID NO: 7, or an amino acid sequence that is at least 80% identical to SEQ ID NO: 9 and polynucleotides encoding these EDDS synthases.

Additionally, the present invention is directed to a method for producing EDDS using a polypeptide having EDDS synthase activity comprising contacting fumaric acid and ethylenediamine with an EDDS synthase under conditions allowing the EDDS synthase to produce EDDS, wherein the EDDS synthase comprises an amino acid sequence that is at least 87% identical to SEQ ID NO: 1, an amino acid sequence that is at least 80% identical to SEQ ID NO: 7, or an amino acid sequence that is at least 80% identical to SEQ ID NO: 9.

Furthermore, the present invention is directed to a method of making the EDDS synthases and a method of producing a composition, preferably a detergent composition, comprising EDDS, wherein the method comprises the steps of a) producing EDDS using the method as claimed herein, and b) formulating the EDDS produced under a) into the composition.

### DETAILED DESCRIPTION OF THE INVENTION

The present invention may be understood more easily by reference to the following definitions, detailed description of the embodiments of the invention and the examples included herein. Although the present invention will be described with respect to particular embodiments, this description is not to be construed in a limiting sense.

### Definitions

Unless otherwise noted, the terms used herein are to be understood according to conventional use by those of ordinary skill in the relevant art.

Before describing exemplary embodiments of the present invention in detail, definitions important for understanding the present invention are provided. Unless stated otherwise or apparent from the nature of the definition, the definitions apply to all compounds, methods and uses described herein.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

Furthermore, the terms “first”, “second”, “third” or “(a)”, “(b)”, “(c)”, “(d)” etc. in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

Throughout this application, various publications are referenced. The disclosures of all these publications and the references cited within those publications in their entireties are hereby incorporated by reference into this application to describe the state of the art to which this invention pertains.

It is to be understood that the term “comprising” is not limiting. For the purposes of the present invention the term “consisting of” is considered to be a preferred embodiment of the term “comprising”. If hereinafter a group is defined to comprise at least a certain number of members, this is meant to also encompass a group which consists of these members only.

In describing the polypeptides of the present invention, the abbreviations for single amino acids are used according to the accepted IUPAC single letter or three letter amino acid abbreviation is used.

“Amino acid alteration” as used herein refers to amino acid substitution, deletion, or insertion.

“Substitutions” are described by providing the original amino acid, followed by the number of the position within the amino acid sequence, followed by the amino acid, which substitutes the original amino acid. For example, the substitution of histidine at position 120 with alanine is designated as “His120Ala” or “H120A”. Substitutions can also be described by merely naming the resulting amino acid without specifying the initial amino acid at this position, e.g., “X120A” or “120A” or “Xaa120Ala” or “120Ala”.

“Deletions” are described by providing the original amino acid followed by the number of the position within the amino acid sequence, followed by *. Accordingly, the deletion of glycine at position 150 is designated as “Gly150*” or “G150*”. Alternatively, deletions are indicated by, e.g., “deletion of D183 and G184”.

“Insertions” are described by providing the original amino acid followed by the number of the position within the amino acid sequence, followed by the original amino acid and the additional amino acid. For example, an insertion at position 180 of lysine next to glycine is designated as “Gly180GlyLys” or “G180GK”. When more than one amino acid residue is inserted, such as, e.g., a Lys and an Ala after Gly180 this may be indicated as: “Gly180GlyLysAla” or “G195GKA”. In cases where a substitution and an insertion occur at the same position, this may be indicated as “S99SD+S99A” or in short “S99AD”. Sequences comprising multiple alterations are separated by “+”, e.g., “Arg170Tyr+Gly195Glu”, “R170Y+G195E” or “X170Y+X195E” representing a substitution of arginine and glycine at positions 170 and 195 with tyrosine and glutamic acid, respectively. Alternatively, multiple alterations may be separated by a space or a comma, e.g., “R170Y G195E” or “R170Y, G195E”, respectively. Where different alternative alterations can be introduced at a position, the different alterations are separated by a comma, e.g., “Arg170Tyr, Glu” and “R170T, E”, respectively, represents a substitution of arginine at position 170 with tyrosine or glutamic acid. Alternative substitutions at a particular position can also be indicated as “X120A,G,H”, “120A,G,H”, “X120A/G/H”, or “120A/G/H”. Alternatively, different alterations or optional substitutions may be indicated in brackets, e.g., “Arg170 [Tyr, Gly]” or “Arg170 {Tyr, Gly}” or in short “R170 [Y, G]” or “R170 {Y, G}”.

The term “native” (or naturally or wildtype or endogenous) cell or organism or polynucleotide or polypeptide refers to the cell or organism or polynucleotide or polypeptide as found in nature (i.e., without there being any human intervention).

The term “isolated” molecule, e. g. polypeptide or polynucleotide, is defined herein as a molecule, which has been separated from its natural environment.

The term “heterologous polypeptide” (or exogenous or foreign polypeptide) is defined herein as a polypeptide, which is naturally not expressed by a host cell. The term “heterologous nucleotide” (or exogenous or foreign polynucleotide) is defined herein as a polynucleotide, which is naturally not contained in a host cell.

For the purpose of the invention, “recombinant” (or non-native or non-naturally) with regards to a cell or an organism means that the cell or organism contains a polynucleotide, which is introduced using gene technology. Recombinant with regards to a polynucleotide or a polypeptide means that the polynucleotide or polypeptide has been newly combined or rearranged in terms of its genetic environment by using recombinant DNA techniques. Thus, recombinant polynucleotides or polypeptides include a polypeptide or polynucleotide native to the host cell, whose expression is quantitatively altered or whose expression is directed from a genomic location different from the native host cell as a result of manipulation of the DNA of the host cell by recombinant DNA techniques, e.g., a stronger promoter. Recombinant polynucleotides or polypeptides can also be heterologous, meaning they can be foreign sequences, but they can also originate from the same organism that they are introduced to.

With respect to the relation between two or more polynucleotides or the relation between two or more polypeptides, the term “recombinant” is used to characterize that the two or more polynucleotides or two or more polypeptides are naturally not occurring in the specific combination with each other.

“Modified recombinant” polynucleotides or polypeptides means recombinant polynucleotides or recombinant polypeptides that have been modified by introducing alterations, e.g., deletions, substitutions, and/or insertions, using recombinant DNA techniques to alter the native polypeptide or native polynucleotide.

A “synthetic” compound is obtained by in vitro chemical and/or enzymatic synthesis.

Sequence identity usually is provided as “% sequence identity” or “% identity”. For calculation of sequence identities, in a first step a sequence alignment is produced. According to this invention, a pairwise global alignment is produced, meaning that two sequences are aligned over their complete length. The alignment is usually produced by using a mathematical approach, called alignment algorithm.

According to the invention, the alignment is generated by using the algorithm of Needleman and Wunsch (J. Mol. Biol. (1979) 48, p. 443-453). Preferably, the program “NEEDLE” (The European Molecular Biology Open Software Suite (EMBOSS)) is used for the purposes of the current invention using the programs default parameter (polynucleotides: gap open=10.0, gap extend=0.5 and matrix=EDNAFULL; polypeptides: gap open=10.0, gap extend=0.5 and matrix=EBLOSUM62). After aligning the two sequences, in a second step, an identity value is determined from the produced alignment. For this purpose, the %-identity is calculated by dividing the number of identical residues by the length of the alignment region, which is showing the respective sequence of the present invention over its complete length multiplied with 100: %-identity=(identical residues/length of the alignment region, which is showing the respective sequence of the present invention over its complete length)*100.

For calculating the percent identity of two polynucleotides the same applies as for the calculation of percent identity as outlined above with some specifications. For polynucleotides encoding for a protein the pairwise alignment shall be made over the complete length of the coding region of the sequence of this invention from start to stop codon excluding introns. Introns present in the other sequence, to which the sequence of this invention is compared to shall also be removed for the pairwise alignment. After aligning the two polynucleotide sequences, in a second step, an identity value is determined from the alignment produced. Percent identity is then calculated by %-identity=(identical residues/length of the alignment region, which is showing the sequence of the invention from start to stop codon excluding introns over its complete length)*100.

Herein, the exchange of one amino acid with a similar amino acid may be called “conservative substitution”. Similar amino acids according to the invention are defined as follows:
Amino acid A is similar to amino acids S
Amino acid D is similar to amino acids E; N
Amino acid E is similar to amino acids D; K; Q
Amino acid F is similar to amino acids W; Y
Amino acid H is similar to amino acids N; Y
Amino acid I is similar to amino acids L; M; V
Amino acid K is similar to amino acids E; Q; R
Amino acid L is similar to amino acids I; M; V
Amino acid M is similar to amino acids I; L; V
Amino acid N is similar to amino acids D; H; S
Amino acid Q is similar to amino acids E; K; R
Amino acid R is similar to amino acids K; Q
Amino acid S is similar to amino acids A; N; T
Amino acid T is similar to amino acids S
Amino acid V is similar to amino acids I; L; M
Amino acid W is similar to amino acids F; Y
Amino acid Y is similar to amino acids F; H; W

A “fragment” or “subsequence” as used herein is a portion of a polynucleotide or an amino acid sequence. The term “functional fragment” refers to any nucleic acid or amino acid sequence which comprises merely a part of the full-length amino acid sequence but still has the same or similar activity and/or function. Preferably, the functional fragment is at least 75%, at least 76%, at least 77%, at least 78%, at least 79%, at least 80% identical, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 99%, or at least 99.5% identical to the original full length amino acid sequence. The functional fragment comprises consecutive nucleic acids or amino acids of the original nucleic acid or original amino acid sequence.

“Genetic construct” or “expression cassette” as used herein, is a nucleic acid molecule composed of at least one sequence of interest to be expressed, operably linked to one or more control sequences (at least to a promoter) as described herein.

The term “vector” as used herein comprises any kind of construct suitable to carry polynucleotide sequences for transfer to a cell, or for stable or transient expression within a given cell. This encompasses any kind of cloning vehicles, such as but not limited to plasmids, phagemids, viral vectors (e.g., phages), bacteriophage, baculoviruses, cosmids, fosmids, artificial chromosomes, and any other vectors specific for specific hosts of interest. Foreign polynucleotide sequences usually comprise a coding sequence which may be referred to herein as “gene of interest”. The gene of interest may comprise introns and exons, depending on the kind of origin or destination of host cell.

The term “introduction of a polynucleotide” or “transformation of a polynucleotide” as referred to herein encompasses the transfer of a polynucleotide outside a host cell into a host cell, irrespective of the method used for transfer. That is, the term “transformation of a polynucleotide” as used herein is independent from vector, shuttle system, or host cell, and it not only relates to the polynucleotide transfer method of transformation as known in the art (cf., for example, Sambrook, J. et al. (1989) Molecular Cloning: A Laboratory Manual, 2nd Ed., Cold Spring Harbor Laboratory Press, Cold Spring Harbor, NY), but it encompasses any further kind of polynucleotide transfer methods such as, but not limited to, transduction or transfection.

A polynucleotide encoding a polypeptide may be “expressed”. The term “expression” or “gene expression” means the transcription of a gene or genes or genetic construct into structural RNA (e.g., rRNA, tRNA) or mRNA with or without subsequent translation of the latter into a protein. The process includes transcription of DNA and processing of the resulting mRNA product.

Cells expressing recombinant polynucleotides or polypeptides may exhibit “increased” or “decreased” expression when compared to the respective wild-type cell. The term “increased expression”, “enhanced expression” or “overexpression” as used herein means any form of expression that is additional to the original wild-type expression level (which can be absence of expression or immeasurable expression as well). Reference herein to “increased expression”, “enhanced expression” or “overexpression” is taken to mean an increase in gene expression and/or, as far as referring to polypeptides, increased polypeptide levels and/or increased polypeptide activity, relative to control organisms. The increase in expression may be at least 5%, at least 10%, at least 20%, at least 30%, at least 40%, at least 50%, at least 60%, at least 70%, at least 80%, at least 85%, at least 90%, or 100% or even more compared to that of the control organism.

The term “purification” or “purifying” refers to a process in which at least one component, e.g., a protein of interest, is separated from at least another component, e.g., a particulate matter of a fermentation broth, and transferred into a different compartment or phase, wherein the different compartments or phases do not necessarily need to be separated by a physical barrier. Examples of such different compartments are two compartments separated by a filtration membrane or cloth, i.e., filtrate and retentate; examples of such different phases are pellet and supernatant or cake and filtrate, respectively. The resulting solution after purifying the enzyme of interest from the fermentation broth is called herein “purified enzyme solution”.

A “composition” is a mixture of components (also called ingredients) prepared according to a specific formula. Compositions include but are not limited to detergent compositions, fertilizer compositions, pharmaceutical compositions, home care compositions or personal care compositions, nutritional supplement compositions, or industrial compositions or formulations such as compositions used in the pulp and paper industry.

“Enzyme properties” include, but are not limited to catalytic activity, substrate/cofactor specificity, product specificity, stability in the course of time, thermostability, pH stability, and chemical stability.

The term “enzyme stability” according to the current invention relates to the retention of enzymatic activity as a function of time during storage or operation. Retention of enzymatic activity as a function of time during storage is called “storage stability” and is preferred within the context of the invention.

To determine and quantify changes in catalytic activity of enzymes stored or used under certain conditions over time, the “initial enzymatic activity” is measured under defined conditions at time zero (100%) and at a certain point later in time (x %). By comparing these values, a potential loss of enzymatic activity can be determined. The extent of loss of enzymatic activity determines an enzyme's stability or non-stability.

“pH stability” refers to the ability of an enzyme to exert enzymatic activity after exposure to certain pH value.

“Detergent composition” or “detergent” means compositions designated for cleaning soiled material. Detergent compositions according to the invention include detergent compositions for different applications such as laundry and hard surface cleaning. The term “detergent component” is defined herein to mean a type of chemical, which can be used in detergent compositions. A typical detergent component is a surfactant. “Surfactant” (synonymously used herein with “surface active agent”) means an organic chemical that, when added to a liquid, changes the properties of that liquid at an interface. According to its ionic charge, a surfactant is called non-ionic, anionic, cationic, or amphoteric. The term “effective amount of a detergent component” includes amounts of certain components to provide effective stain removal and/or effective cleaning conditions (e. g. pH, temperature, water hardness), amounts of certain components to effectively provide optical benefits (e. g. optical brightening, dye transfer inhibition, color care), and amounts of certain components to effectively aid the processing (maintain physical characteristics during processing, storage and use; e.g. rheology modifiers, hydrotropes, desiccants).

The term “hard surface cleaning” relates to both household hard surface cleaning and industrial hard surface cleaning and means the process of treating hard surfaces with a solution containing a detergent composition of the present invention. Hard surfaces may include any hard surfaces in the household or industry, such as floors, furnishing, walls, sanitary ceramics, glass, metallic surfaces including medical devices, cutlery, and dishes. A particular form of hard surface cleaning is dishwashing, including manual dish washing (MDW) or automatic dishwashing (ADW).

The term “dish wash” refers to all forms of washing dishes, e.g. by hand or automatic dish washing. Washing dishes includes, but is not limited to, the cleaning of all forms of crockery such as plates, cups, glasses, bowls, all forms of cutlery such as spoons, knives, forks and serving utensils as well as ceramics, plastics such as melamine, metals, china, glass and acrylics.

The term “medical device cleaning” refers to the cleaning step in reprocessing reusable medical devices. Medical device cleaning methods can be divided into two categories, manual and mechanical/automated cleaning methods. Manual cleaning is used when mechanical units are not available or medical devices to be cleaned are too fragile or difficult to clean with a mechanical unit. Mechanical/automated cleaning methods remove soiling and microorganisms through an automated cleaning and rinsing process, this includes ultrasonic cleaning and washing.

A composition “essentially devoid” of a compound shall mean herein that the respective compound is not added to the composition on purpose, meaning that at most non-effective amounts are present, most preferably 0% of the compound are contained in the composition.

### DETAILED DESCRIPTION

In the present invention new EDDS synthases are provided. More specifically, new polypeptides having EDDS synthase activity, methods of using the EDDS synthases to produce EDDS, methods of producing a composition comprising EDDS and methods of making the EDDS synthases are provided.

### EDDS Synthases

The present invention is directed to EDDS synthases. EDDS synthases according to the invention have “EDDS synthase activity”. “EDDS synthase activity” describes the capability of catalyzing the reaction of EDDS formation using one molecule of ethylenediamine and two molecules of fumaric acid as substrates. EDDS synthase activity may be determined as described in the literature (e. g. by Poddar et al., Biochemistry 2018, 57, 26, 3752-3763, by Wang et al., Process Biochemistry 2022, 116, 38-48, or by Takahashi et al., Biosci. Biotechnol. Biochem 1999, 63 (7), 1269-1273). For instance, EDDS synthase activity can be determined by using ethylenediamine and fumaric acid as substrates and analyzing the synthesized EDDS using HPLC measurements.

As a side note, EDDS synthases have also been shown, e.g. by Poddar et al., Biochemistry 2018, 57, 26, 3752-3763, to accept a wide variety of different mono- and diamine substrates instead of ethylenediamine together with fumarate to generate different aminocarboxylic acid products, including EDDS derivatives.

The EDDS synthase of the present invention is an EDDS synthase having an amino acid sequence that is at least 87% identical to SEQ ID NO: 1, an amino acid sequence that is at least 80% identical to SEQ ID NO: 7, or an amino acid sequence that is at least 80% identical to SEQ ID NO: 9.

In one embodiment, the EDDS synthase comprises an amino acid sequence that is with increasing preference at least 87%, at least 88%, at least 89%, at least 90%, at least 91%, at least 92%, at least 93%, at least 94%, at least 95%, at least 96%, at least 97%, at least 98%, at least 99% or 100% identical to SEQ ID NO: 1.

In another embodiment, the EDDS synthase comprises an amino acid sequence that is with increasing preference at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to SEQ ID NO: 7.

In another embodiment, the EDDS synthase comprises an amino acid sequence that is with increasing preference at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to SEQ ID NO: 9.

Preferably, the EDDS synthase comprises an amino acid sequence that is with increasing preference at least 90%, at least 91%, at least 92%, at least 93%, at least 94%, at least 95%, at least 96%, at least 97%, at least 98%, at least 99% or 100% identical to SEQ ID NO: 1, 7 or 9.

More preferably, the EDDS synthase comprises an amino acid sequence that is with increasing preference at least 90%, at least 91%, at least 92%, at least 93%, at least 94%, at least 95%, at least 96%, at least 97%, at least 98%, at least 99% or 100% identical to SEQ ID NO: 1 or 7.

Most preferably, the EDDS synthase comprises an amino acid sequence that is with increasing preference at least 90%, at least 91%, at least 92%, at least 93%, at least 94%, at least 95%, at least 96%, at least 97%, at least 98%, at least 99% or 100% identical to SEQ ID NO: 1.

In one embodiment, the EDDS synthase consists of an amino acid sequence that is 100% identical to SEQ ID NO: 1, 7, or 9, preferably to SEQ ID NO: 1.

In one embodiment, the EDDS synthase comprises or consists of an amino acid sequence that is 100% identical to SEQ ID NO: 1, 7, or 9, preferably SEQ ID NO: 1, but comprising 1-20, preferably 1-15, more preferably 1-10, even more preferably 1-5 conservative amino acid substitutions.

Ways of introducing amino acid alterations, e.g., substitutions, preferably conservative substitutions, into protein sequence are well known in the art. Substitutions can be introduced by using any mutagenesis procedure known in the art, such as site-directed mutagenesis, synthetic gene construction, semi-synthetic gene construction, random mutagenesis, shuffling, etc followed by a relevant screening procedure. See, e.g., Scherer and Davis, 1979, Proc. Nat/. Acad. Sci. USA 76: 4949-4955; and Barton et al., 1990, Nucleic Acids Res. 18: 7349-4966, U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16, Tian et al, 2004, Nature 432: 1050-1054; Reidhaar-Olson and Sauer, 1988, Science 241: 53-57; Bowie and Sauer, 1989, Proc. Nat/. Acad. Sci. USA 86: 2152-2156; WO 95/17413; or WO 95/22625, Lowman et al., 1991, Biochemistry 30: 10832-10837; U.S. Pat. No. 5,223,409; WO 92/06204, Derbyshire et al., 1986, Gene 46: 145; Ner et al., 1988, DNA 7: 127.

In a preferred embodiment, the EDDS synthase of the present invention possesses a specific activity (kU/L*signal factor) above 1.5, preferably above 2.0, further preferred above 2.5, most preferred above 3.0, wherein the signal factor is the SDS page signal intensity relative to the intensity of SEQ ID NO: 1.

In a preferred embodiment, the EDDS synthase of the present invention shows highest EDDS synthase activity in a temperature range between 20° C. and 40° C., preferably between 30° C. and 40° C., more preferably between 35° C. and 40° C.

In a preferred embodiment, the EDDS synthase of the present invention shows EDDS synthase activity in a pH range between 6 and 10, preferably between 7 and 9, more preferably between 8 and 9.

In one embodiment, the EDDS synthase of the present invention is a purified, isolated, synthetic, and/or recombinant EDDS synthase. Preferably, the EDDS synthase of the present invention is a recombinant EDDS synthase.

### Nucleic Acid Construct

The present invention also refers to a polynucleotide encoding the EDDS synthase of the present invention or the complement thereof. Preferably, the polynucleotide is a codon-optimized polynucleotide for improving expression in a specific host cell, preferably an E. coli cell.

The present invention thus also refers to a nucleic acid, preferably an isolated, a synthetic, and/or a recombinant nucleic acid comprising:
(a) a polynucleotide having with increasing preference at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 2, SEQ ID NO: 8, or SEQ ID NO: 10, wherein the nucleic acid encodes a polypeptide having EDDS synthase activity;
(b) a polynucleotide encoding a polypeptide having with increasing preference at least 87%, at least 90%, at least 92%, at least 95%, at least 97%, or 100% identity to SEQ ID NO: 1, or at least 80%, at least 82%, at least 85%, at least 87%, at least 90%, at least 92%, at least 95%, at least 97%, or 100% identity to SEQ ID NO: 7, or at least 80%, at least 82%, at least 85%, at least 87%, at least 90%, at least 92%, at least 95%, at least 97%, or 100% identity to SEQ ID NO: 9, wherein the polypeptide has EDDS synthase activity;
(c) a polynucleotide that hybridizes under high stringency conditions, preferably under very high stringency conditions, with the complement of
   (i) a coding sequence of SEQ ID NO: 1, SEQ ID NO: 7, or SEQ ID NO: 9; or
   (ii) a polynucleotide shown in SEQ ID NO: 2, SEQ ID NO: 8, or SEQ ID NO: 10;
(d) a fragment of (a), (b), or (c), wherein the fragment encodes a polypeptide having EDDS synthase activity; or
(e) a polynucleotide fully complementary to any of (a) to (d).
(f) a polynucleotide that differs from any of the polynucleotides described in (a) to (e) merely by the degeneracy of the genetic code.

In a further embodiment, the invention also relates to polypeptides having EDDS synthase activity, which are encoded by a polynucleotide described herein, preferably isolated, synthetic or recombinant polypeptides having EDDS synthase activity, which are encoded by a polynucleotide described herein. Preferably, the polypeptide having EDDS synthase activity is encoded by a polynucleotide having with increasing preference at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 2, SEQ ID NO: 8, or SEQ ID NO: 10.

In another embodiment, the polypeptide having EDDS synthase activity is encoded by a polynucleotide that hybridizes under high stringency conditions, preferably under very high stringency conditions, with the full-length complement of a polynucleotide having with increasing preference at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% identity to SEQ ID NO: 2, SEQ ID NO: 8, or SEQ ID NO: 10.

The present invention also refers to a nucleic acid construct, preferably an expression cassette, comprising the polynucleotide as described herein.

Typically, the expression cassette comprises three elements: a promoter sequence, an open reading frame, and a 3′ untranslated region that, in eukaryotes, usually contains a polyadenylation site. Additional regulatory elements may include transcriptional as well as translational enhancers. An intron sequence may also be added to the 5′ untranslated region (UTR) or in the coding sequence to increase the amount of the mature messenger RNA that accumulates in the cytosol. The expression cassette may be part of a vector or may be integrated into the genome of a host cell and replicated together with the genome of its host cell. The expression cassette is usually capable of increasing or decreasing expression.

The present invention also refers to an expression vector comprising the polynucleotide, or the nucleic acid construct as described herein. The expression vector can be a low copy number vector or high copy number vector.

A vector as used herein may provide segments for transcription and translation of a polynucleotide upon transformation into a host cell or host cell organelles. Such additional segments may include regulatory nucleotide sequences, one or more origins of replication that is required for its maintenance and/or replication in a specific cell type, one or more selectable markers, a polyadenylation signal, a suitable site for the insertion of foreign coding sequences such as a multiple cloning site etc. One example is when a vector is required to be maintained in a bacterial cell as an episomal genetic element (e.g., plasmid or cosmid molecule). Non-limiting examples of suitable origins of replication include the f1-ori and colE1.

A vector may replicate without integrating into the genome of a host cell, e.g., as a plasmid in a bacterial host cell, or it may integrate part of or all its DNA into the genome of the host cell and thus lead to replication and expression of its DNA.

The polynucleotide encoding the EDDS synthase of the present invention may be introduced into a vector by means of standard recombinant DNA techniques. Once introduced into the vector, the polynucleotide comprising a coding sequence may be suitable to be introduced (transformed, transduced, transfected, etc.) into a host cell or host cell organelles. A cloning vector suitable for expression of the polynucleotide sequence in the host cell or host cell organelles may be chosen.

### Host Cell

The present invention also refers to a host cell comprising the polynucleotide encoding the EDDS synthase as described herein, the nucleic acid construct as described herein, or the expression vector as described herein. In one embodiment of the invention, a vector is used for transformation of a host cell.

The polynucleotide encoding the EDDS synthase as described herein may be transiently or stably introduced into a host cell and may be maintained non-integrated, for example, as a plasmid. Usually, stable transformation is due to integration of a nucleic acid comprising a recombinant coding sequence into the chromosomes or as an episome (separate piece of nuclear DNA). Usually, transient transformation is due to a nucleic acid comprising a recombinant nucleic acid sequence not being integrated into the chromosomes or as an episome.

The introduction of a nucleic acid into a host cell may, for instance, but not limited thereto, be effected by protoplast transformation (see, e.g., Chang and Cohen, 1979, Molecular General Genetics 168: 111-115), by using competent cells (see, e.g., Young and Spizizen, 1961, Journal of Bacteriology 81: 823-829, or Dubnau and Davidoff-Abelson, 1971, Journal of Molecular Biology 56: 209-221), by electroporation (see, e.g., Shigekawa and Dower, 1988, Biotechniques 6: 742-751), or by conjugation (see, e.g., Koehler and Thorne, 1987, Journal of Bacteriology 169: 5271-5278). Specific transformation protocols are known in the art for various types of host cells (see, e.g., for E. coli protoplast transformation see Hanahan, 1983, J. Mol. Biol. 166: 557-580). Various host cells can be used for expressing the nucleic acid construct described herein. Host cells comprising the genetic constructs described herein can be obtained by one of the methods described herein for introducing the polynucleotides into such host cells. The host cell of the present invention does not naturally express the EDDS synthase. Thus, the host cell is a recombinant host cell and the nucleic acid construct described herein is heterologous for the host cell.

In one embodiment, the host cell is a prokaryote or a eukaryote. Preferably, the host cell is a bacterial cell, an archaeal cell, a fungal cell, a yeast cell, insect cell, mammalian cell, or any other eukaryotic cell. In another embodiment, the host cell is a non-human host cell.

In one embodiment, the host cell is a bacterial cell. The bacterial host cell may be any gram-positive bacterium or a gram-negative bacterium. Gram-positive bacteria include, but are not limited to, Bacillus, Brevibacterium, Corynebacterium, Streptococcus, Streptomyces, Staphylococcus, Enterococcus, Lactobacillus, Lactococcus, Clostridium, Paenibacillus, Geobacillus, and Oceanobacillus. Gram-negative bacteria include, but are not limited to, Escherichia, Pseudomonas, Salmonella, Campylobacter, Basfia, Ensifer, Sinorhizobium, Helicobacter, Acetobacter, Flavobacterium, Fusobacterium, Gluconobacter. In one embodiment, the host cell is a bacterial cell. In a specific embodiment the host cell is of the genus Escherichia or Bacillus. In a specific embodiment, the bacterial host cell is an Escherichia cell. In a preferred embodiment, the bacterial host cell is an Escherichia coli cell.

In the methods of the present invention, the bacterial host cell may be a Bacillus cell. Bacillus cells useful in the practice of the present invention include, but are not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus methylotrophicus, Bacillus cereus, Bacillus paralicheniformis, Bacillus subtilis, and Bacillus thuringiensis cells. In one embodiment, the bacterial host cell is a Bacillus amyloliquefaciens, Bacillus pumilus, Bacillus lentus, Bacillus licheniformis, Bacillus stearothermophilus or Bacillus subtilis cell. In preferred embodiment, the bacterial host cell is a Bacillus licheniformis cell, a Bacillus pumilus, or a Bacillus subtilis cell. Preferably, the bacterial host cell is a Bacillus licheniformis cell.

In the methods of the present invention, the bacterial host cell may be Lactobacillus acidophilus, Lactobacillus plantarum, Lactobacillus gasseri, Lactobacillus bulgaricusk, Lactobacillus reuteri, Staphylococcus aureus, Corynebacterium glutamicum, Corynebacterium acetoglutamicum, Corynebacterium acetoacidophilum, Corynebacterium callunae, Corynebacterium ammoniagenes, Corynebacterium thermoaminogenes, Corynebacterium melassecola, Corynebacterium effiziens, Corynebacterium efficiens, Corynebacterium deserti, Brevibacterium flavum, Brevibacterium lactofermentum, Brevibacterium divarecatum, Pseudomonas putida, Pseudomonas syringae, Streptomyces coelicolor, Streptomyces lividans, Streptomyces albus, Streptomyces avermitilis, Gluconobacter oxydans, Gluconobacter morbifer, Gluconobacter thailandicus, Acetobacter aceti, Clostridium acetobutylicum, Clostridium saccharobutylicum, Clostridium beijerinckii, Streptococcus equisimilis, Streptococcus pyogenes, Streptococcus uberis, Streptococcus equi subsp., Zooepidemicus or Basfia succiniciproducens.

In another embodiment, the bacterial host cell may additionally contain modifications, e.g., deletions or disruptions, of other genes that may be detrimental to the production, recovery or application of a polypeptide of interest.

In one embodiment, the bacterial host cell is a host cell with reduced fumarase activity, preferably a fumarase-deficient cell. The reduction in or deletion of the fumarase activity can occur naturally in the host cell or can be achieved by gene technology. In a more specific embodiment, the bacterial host cell is a fumarase-deficient E. coli cell. In another embodiment, the bacterial host cell, e.g., E. coli cell, comprises a disruption and/or deletion of the fumarase genes fumA, B and/or C. FumA, B and/or C genes are described for example by Derbikov, D. D. et al., 2017, Appl Biochem Microbiol 53, 859-866 or Zhang et al., Appl Microbiol Biotechnol. 2015 June; 99(12):5033-44. Without wishing to be bound to theory, the reduction or deletion of fumarase activity in the host cell, preferably E. coli, leads to a reduced concentration of by-products, such as maleic acid compared to a host cell with no reduction or deletion of the fumarase activity.

In an alternative embodiment, the host cell does not comprise a reduced or deleted fumarase activity.

In another embodiment, is a host cell comprises a genetic construct encoding one or more chaperones capable of assisting the correct folding of the EDDS synthase. In a preferred embodiment, the host cell is a bacterial host cell, preferably an E. coli cell, which comprises a genetic construct encoding a GroEL/GroES protein complex. The genetic construct encoding the GroEL/GroES complex can be located on the same vector or can be located on a different vector than the construct encoding the EDDS synthase as described herein.

In another embodiment, the bacterial host cell is a standard E. coli host cell, which is used for cloning or protein expression, including but not limited to DH5alpha (Invitrogen), DH10B, (Invitrogen), Omnimax (Invitrogen), INV110 (Invitrogen), TOP10 (Invitrogen), HB101 (Promega), SURE (Stratagene), XL1-Blue (Stratagene), TG1 (Lucigen), BL21 and its variants (Invitrogen), and JM109 (NEB). In another embodiment, variants and derivatives of the listed E. coli cells, such as the E. coli strain TG 10 (see experimental description for strain details) are used. In a particularly preferred embodiment, the bacterial host cell is an E. coli TG10 cell. In another embodiment, the bacterial host cell is a standard Bacillus subtilis cloning host cell, including but not limited to B. subtilis carrying a defective hsd(RI)R-M-locus such as B. subtilis IG-20 (BGSC 1A436) or a defective hsdRM1 mutation such as B. subtilis 1012 WT (Mobitec).

Alternative further host cells include but are not limited to: Aspergillus niger, Aspergillus oryzae, Hansenula polymorpha, Thermomyces lanuginosus, Fusarium oxysporum, Fusarium heterosporum, Pichia pastoris (also known as Komagataella phaffii), Myceliopthora thermophile (C1), Themothelomyces thermophila, Schizosaccharomyces pombe, Trichoderma, preferably Trichoderma reesei and Saccharomyces, preferably Saccharomyces cerevisiae, or Rhizomucor.

### Methods of Making the EDDS Synthase

The EDDS synthase of the present invention can be produced by any method for protein synthesis known in the art. Preferably, the EDDS synthase of the present invention is produced in an industrial scale. Industrial production of enzymes usually is done by cultivation of a host cell (also called fermentation) which expresses the enzyme. Suitable host cells are described herein. A polynucleotide encoding the EDDS synthase described herein can be transformed into the host cell, which is subsequently cultivated under conditions suitable for the host cell to produce the EDDS synthase of the present invention.

Hence, in yet another embodiment, the present invention is directed to a method of producing an EDDS synthase described herein, comprising the steps of
(a) providing a host cell comprising a recombinant nucleic acid construct comprising a polynucleotide encoding the EDDS synthase described herein by introducing the nucleic acid construct comprising the polynucleotide encoding the EDDS synthase described herein into the host cell;
(b) cultivating the recombinant host cell of step (a) under conditions conductive for the expression of the polynucleotide; and
(c) optionally, recovering the EDDS synthase encoded by the polynucleotide.

Preferably, the host cell used for making the EDDS synthase is an E. coli cell, preferably an E. coli cell, which comprises a genetic construct encoding one or more chaperones, preferably a GroEL/GroES protein complex. In one embodiment, the E. coli cell is having a reduction or deletion of fumarase activity.

Cultivation of the host cell normally takes place in a suitable nutrient medium allowing the recombinant cells to grow and express the desired protein. At the end of fermentation, the fermentation broth is collected and may be further processed, wherein the fermentation broth comprises a liquid fraction and a solid fraction. The enzyme of interest may be further purified from the fermentation broth.

The EDDS synthase described herein may be secreted (into the liquid fraction of the fermentation broth) or may not be secreted from the microbial cells (and therefore is comprised in the cells of the fermentation broth). Depending on this, the EDDS synthase may be recovered from the liquid fraction of the fermentation broth or from the host cell or the recovered cells might be used as whole cell catalysts using methods known in the art. Recovery of the EDDS synthase can be achieved by methods known to those skilled in the art. Suitable methods for recovery of proteins from fermentation broth include but are not limited to collection, centrifugation, filtration, extraction, spray-drying, evaporation, or precipitation. The isolated polypeptide may then be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989). The purified polypeptide may then be concentrated by procedures known in the art including, but not limited to, ultrafiltration and evaporation, in particular thin film evaporation. If the product of interest precipitates or crystallizes in the fermentation broth or binds at least in part to the particulate matter of the fermentation broth additional treatment steps might be needed to release the protein of interest from the biomass or to solubilize protein of interest crystals and precipitates. In case the desired protein is comprised in the cells of the fermentation broth and is supposed to be released, release of the protein of interest from the cells might be needed. Release from the cells can be achieved for instance, but not being limited thereto, by cell lysis with techniques well known to the skilled person, e.g., lysozyme treatment, ultrasonic treatment, French press or combinations thereof.

### Formulation or Enzyme Preparation Comprising EDDS Synthase

The present invention is also directed to a formulation comprising a polypeptide having EDDS synthase activity and at least one additional component, preferably the additional compound is selected from the group consisting of solvent, enzyme stabilizing compound and preservative, wherein the polypeptide having EDDS synthase activity comprises an amino acid sequence that is at least 87% identical to SEQ ID NO: 1, an amino acid sequence that is at least 80% identical to SEQ ID NO: 7, or an amino acid sequence that is at least 80% identical to SEQ ID NO: 9.

### Methods of Producing EDDS

The present invention is also directed to a method for producing EDDS using a polypeptide having EDDS synthase activity comprising contacting fumaric acid and ethylenediamine with an EDDS synthase under conditions allowing the EDDS synthase to produce EDDS, wherein the EDDS synthase comprises an amino acid sequence that is at least 87% identical to SEQ ID NO: 1, an amino acid sequence that is at least 80% identical to SEQ ID NO: 7, or an amino acid sequence that is at least 80% identical to SEQ ID NO: 9.

The EDDS produced by the methods described herein can be analyzed using HPLC measurements.

For the methods for producing EDDS as described herein, the EDDS synthase can be purified and used as a catalyst in its pure form or whole cells expressing the EDDS synthase can be used as whole cell catalysts.

In a preferred embodiment, the EDDS is produced using a whole cell catalyst, which expresses the EDDS synthase described herein. Preferably, the whole cell catalyst is a host cell as described herein.

Thus, in one embodiment the present invention is directed to a method for producing EDDS comprising the steps of
a) providing a host cell comprising a recombinant nucleic acid construct comprising a polynucleotide encoding the EDDS synthase described herein by introducing the nucleic acid construct comprising the polynucleotide encoding the EDDS synthase described herein into the host cell;
b) cultivating the host cell of step (a) under conditions conductive for the expression of the polynucleotide encoding the EDDS synthase;
c) optionally harvesting the host cell step (b) and
d) contacting the host cell of step (b) and/or (c) with fumaric acid and ethylenediamine under conditions allowing the fumaric acid and ethylenediamine to be in contact with the EDDS synthase to produce EDDS;
   wherein the EDDS synthase comprises an amino acid sequence that is at least 87% identical to SEQ ID NO: 1, an amino acid sequence that is at least 80% identical to SEQ ID NO: 7, or an amino acid sequence that is at least 80% identical to SEQ ID NO: 9.

Preferably the whole cell catalyst is an E. coli cell expressing the EDDS synthase described herein. Preferably, the host cell, preferably the E. coli host cell, comprises a genetic construct encoding one or more chaperones, preferably a GroEL/GroES protein complex, as described herein. Preferably, the host cell, preferably the E. coli host cell, comprises a reduced or deleted fumarase activity as described herein. In an alternative embodiment, the host cell does not comprise a reduced or deleted fumarase activity and the method of making EDDS using the EDDS synthase of the present invention includes inactivation of fumarase side activity by heat treatment or alkaline treatment.

Thus, in another embodiment, the present invention is directed to a method for producing EDDS comprising the steps of
a) providing a host cell comprising a recombinant nucleic acid construct comprising a polynucleotide encoding the EDDS synthase described herein by introducing the nucleic acid construct comprising the polynucleotide encoding the EDDS synthase described herein into the host cell;
b) cultivating the recombinant host cell of step (a) under conditions conductive for the expression of the polynucleotide;
c) optionally harvesting the host cell of step (b);
d) exposing the host cell obtained in step (b) and/or (c) to conditions suitable for inactivation of fumarase side activity of the host cell, e.g, by heat treatment or alkaline treatment, and
e) contacting the host cell of step (d) with fumaric acid and ethylenediamine under conditions allowing the fumaric acid and ethylenediamine to be in contact with the EDDS synthase to produce EDDS
   wherein the EDDS synthase comprises an amino acid sequence that is at least 87% identical to SEQ ID NO: 1, an amino acid sequence that is at least 80% identical to SEQ ID NO: 7, or an amino acid sequence that is at least 80% identical to SEQ ID NO: 9.

Chelating agents such as EDDS and their respective alkali metal salts are useful sequestrants for metal ions (e. g. metal ions such as Mg2+, Ca2+, Ba2+, Cu2+, Fe2+, Zn2+, Fe3+). Therefore, in one embodiment, the method for producing EDDS using a polypeptide having EDDS synthase activity comprises contacting fumaric acid and ethylenediamine and additionally a metal ion, preferably a metal salt, more preferably magnesium, most preferably magnesium hydroxide, with an EDDS synthase under conditions allowing the EDDS synthase to produce EDDS, wherein the EDDS synthase comprises an amino acid sequence that is at least 87% identical to SEQ ID NO: 1, an amino acid sequence that is at least 80% identical to SEQ ID NO: 7, or an amino acid sequence that is at least 80% identical to SEQ ID NO: 9. In a preferred embodiment, the EDDS synthesized using the EDDS synthase of the present invention, is in the form of a metal-EDDS complex, preferably in the form of an Mg-EDDS complex.

EDDS possesses two chiral centers, leading to the general possibility of generating three stereoisomeric forms, namely (R,R)-EDDS, (S,S)-EDDS and achiral meso-(R,S)-EDDS.

In a preferred embodiment, the EDDS synthase of the present invention produces predominantly (S,S)-EDDS. In a preferred embodiment, the EDDS synthase of the present invention produces (S,S)-EDDS with optical purity exceeding at least 90%, at least 91%, at least 92%, at least 93%, at least 94%, at least 95%, at least 96%, at least 97%, at least 98%, at least 99% enantiomeric excess (ee) or 100% (S,S)-EDDS.

In a preferred embodiment, the (S,S)-EDDS synthesized by the EDDS synthase of the present invention is biodegradable.

Therefore, in a preferred embodiment, the method for producing EDDS using a polypeptide having EDDS synthase activity comprises contacting fumaric acid and ethylenediamine with an EDDS synthase under conditions allowing the EDDS synthase to produce (S,S)-EDDS, wherein the EDDS synthase comprises an amino acid sequence that is at least 87% identical to SEQ ID NO: 1, an amino acid sequence that is at least 80% identical to SEQ ID NO: 7, or an amino acid sequence that is at least 80% identical to SEQ ID NO: 9 and wherein the optical purity of the (S,S)-EDDS produced has with increasing preference at least 90%, at least 91%, at least 92%, at least 93%, at least 94% at least 95%, at least 96%, at least 97%, at least 98%, at least 99% enantiomeric excess (ee) or is 100% (S,S)-EDDS.

In a preferred embodiment, the method step of producing EDDS using a polypeptide having EDDS synthase activity as described herein is performed in a temperature range between 20° C. and 40° C., preferably between 30° C. and 40° C., more preferably between 35° C. and 40° C.

In a further preferred embodiment, the method step of producing EDDS using a polypeptide having EDDS synthase activity as described herein is performed in a pH range between 6 and 10, preferably between 7 and 9, more preferably between 8 and 9.

In an alternative embodiment, the present invention also refers to a method of producing an EDDS derivative using a polypeptide having EDDS synthase activity comprising contacting fumaric acid and ethylenediamine derivative, which is mono- or diamine substrate different to EDDS, with an EDDS synthase under conditions allowing the EDDS synthase to produce the EDDS derivative, wherein the EDDS synthase comprises an amino acid sequence that is at least 87% identical to SEQ ID NO: 1, an amino acid sequence that is at least 80% identical to SEQ ID NO: 7, or an amino acid sequence that is at least 80% identical to SEQ ID NO: 9.

### Methods of Usinq EDDS

The EDDS obtained by any of the methods described herein can be used in a composition. The composition can in principle be any composition. Preferably the composition is a composition in which a chelating agent provides a beneficial effect. Preferably, EDDS produced by any of the methods described herein is used in the composition as builder.

Compositions comprising EDDS obtained by any of the methods described herein include but are not limited to a detergent composition, a fertilizer composition, a pharmaceutical composition, home care compositions or personal care compositions, a nutritional supplement composition, a composition suitable for producing pulp or compositions used in the paper industry, in photographic supplies, for soil remediation, and in wastewater treatment.

Most preferably, the EDDS obtained by any of the methods described herein can be used in a detergent composition. The detergent composition preferably is a laundry or hard surface cleaning detergent composition, suitable for home care and/or industrial and institutional (I&I) cleaning. Both the laundry and the hard surface cleaning composition can be in the form of a hand wash or an automated wash composition. The hard surface cleaning detergent composition can preferably be a dish wash composition or a medical instrument cleaning composition. Preferably, the dish wash composition is an Automatic Dish Wash (ADW) composition.

### Methods of Producing a Composition Comprising EDDS

In one embodiment, the present invention is directed to a method of producing a composition comprising EDDS, wherein the method comprises the steps of:
a) producing EDDS using the method for producing EDDS using a polypeptide having EDDS synthase activity comprising contacting fumaric acid and ethylenediamine with an EDDS synthase under conditions allowing the EDDS synthase to produce EDDS, wherein the EDDS synthase comprises an amino acid sequence that is at least 87% identical to SEQ ID NO: 1, an amino acid sequence that is at least 80% identical to SEQ ID NO: 7, or an amino acid sequence that is at least 80% identical to SEQ ID NO: 9 as described herein, and
b) formulating the EDDS produced under a) into a composition. In a preferred embodiment, the present invention is directed to a method of producing a detergent composition comprising EDDS, wherein the method comprises the steps of:
a) producing EDDS using the method for producing EDDS using a polypeptide having EDDS synthase activity comprising contacting fumaric acid and ethylenediamine with an EDDS synthase under conditions allowing the EDDS synthase to produce EDDS, wherein the EDDS synthase comprises an amino acid sequence that is at least 87% identical to SEQ ID NO: 1, an amino acid sequence that is at least 80% identical to SEQ ID NO: 7, or an amino acid sequence that is at least 80% identical to SEQ ID NO: 9 as described herein, and
b) formulating the EDDS produced under a) into a detergent composition.

In one embodiment, the step of b) formulating the EDDS produced under a) into a composition comprises the steps of formulating
b1) EDDS as produced by the method described herein; and
b2) one or more components different to EDDS, preferably a detergent component as described herein, into a composition.

The one or more detergent component may be selected from the group consisting of an additional chelating agent and/or builder different to EDDS, an enzyme, an enzyme stabilizing system, a surfactant, a defoamer, a polymer, a bleaching system (bleach), a rheology modifier, a hydrotrope, a softening agent, a desiccant, a whitening agent, a buffer, a preservative, an anticorrosion additive, a dyestuff and a fragrance. Preferably the one or more of the detergent components is bio-degradable and/or bio-based.

### Preferred Embodiments

Particularly, preferred herein is:
1. A method for producing EDDS using a polypeptide having ethylenediamine-N,N′-disuccinic acid (EDDS) synthase activity comprising contacting fumaric acid and ethylenediamine with an EDDS synthase under conditions allowing the EDDS synthase to produce EDDS, wherein the EDDS synthase comprises an amino acid sequence that is at least 87% identical to SEQ ID NO: 1, an amino acid sequence that is at least 80% identical to SEQ ID NO: 7, or an amino acid sequence that is at least 80% identical to SEQ ID NO: 9.
2. The method for producing EDDS according to embodiment 1, wherein the EDDS synthase comprises with increasing preference at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 1.
3. The method for producing EDDS according to embodiment 1, wherein the EDDS synthase comprises with increasing preference at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 7.
4. The method for producing EDDS according to embodiment 1, wherein the EDDS synthase comprises with increasing preference at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 9.
5. The method for producing EDDS according to embodiment 1, wherein the EDDS synthase comprises with increasing preference at least 90%, at least 91%, at least 92%, at least 93%, at least 94%, at least 95%, at least 96%, at least 97%, at least 98%, at least 99% or 100% identity to SEQ ID NO: 1, SEQ ID NO: 7, or SEQ ID NO: 9, more preferably to SEQ ID NO: 1 or to SEQ ID NO: 7, most preferably to SEQ ID NO: 1.
6. The method for producing EDDS according to any of the preceding embodiments, wherein the step of contacting fumaric acid and ethylenediamine with the EDDS synthase under conditions allowing the EDDS synthase to produce EDDS is conducted in the presence of magnesium hydroxide, preferably wherein the produced EDDS chelates magnesium to form Mg-EDDS.
7. The method for producing EDDS according to any of the preceding embodiments, wherein (S,S)-EDDS is produced and wherein the optical purity of the produced (S,S)-EDDS has with increasing preference at least 90%, at least 91%, at least 92%, at least 93%, at least 94%, at least 95%, at least 96%, at least 97%, at least 98%, at least 99% enantiomeric excess (ee) or is 100% (S,S)-EDDS.
8. The method for producing EDDS according to any of the preceding embodiments, wherein the step of producing EDDS using a polypeptide having EDDS synthase activity is performed in a temperature range between 20° C. and 40° C., preferably between 30° C. and 40° C., more preferably between 35° C. and 40° C.
9. The method for producing EDDS according to any of the preceding embodiments, the step of producing EDDS using a polypeptide having EDDS synthase activity is performed in a pH range between 6 and 10, preferably between 7 and 9, more preferably between 8 and 9.
10. An isolated, synthetic, or recombinant polypeptide having EDDS synthase activity, wherein the recombinant polypeptide having EDDS synthase activity comprises with increasing preference at least 88%, at least 89%, at least 90%, at least 91%, at least 92%, at least 93%, at least 94%, at least 95%, at least 96%, at least 97%, at least 98%, at least 99% or 100% identity to SEQ ID NO: 1.
11. An isolated, synthetic, or recombinant polypeptide having EDDS synthase activity, wherein the recombinant polypeptide having EDDS synthase activity comprises with increasing preference at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 7.
12. An isolated, synthetic, or recombinant polypeptide having EDDS synthase activity, wherein the recombinant polypeptide having EDDS synthase activity comprises with increasing preference at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity to SEQ ID NO: 9.
13. An isolated, synthetic, or recombinant polypeptide having EDDS synthase activity, wherein the recombinant polypeptide having EDDS synthase activity comprises with increasing preference at least 90%, at least 91%, at least 92%, at least 93%, at least 94%, at least 95%, at least 96%, at least 97%, at least 98%, at least 99% or 100% identity to SEQ ID NO: 1, SEQ ID NO: 7, or SEQ ID NO: 9, more preferably to SEQ ID NO: 1 or to SEQ ID NO: 7, most preferably to SEQ ID NO: 1.
14. The isolated, synthetic, or recombinant polypeptide according to any of preferred embodiments 8 to 11, wherein the polypeptide comprises or consists of SEQ ID NO: 1, SEQ ID NO: 7, or SEQ ID NO: 9, more preferably SEQ ID NO: 1 or SEQ ID NO: 7, most preferably SEQ ID NO: 1, or wherein the amino acid sequence of the polypeptide having EDDS synthase activity comprises or consists of SEQ ID NO: 1, SEQ ID NO: 7, or SEQ ID NO: 9, more preferably SEQ ID NO: 1 or SEQ ID NO: 7, most preferably SEQ ID NO: 1, with 1-20 amino acid substitutions, preferably 1-10 or more preferably 1-5 amino acid substitutions, preferably wherein the substitutions are conservative substitutions.
15. A method of making the isolated, synthetic, or recombinant polypeptide of any of preferred embodiments 10 to 14, comprising the steps of a) providing a polynucleotide encoding the polypeptide having EDDS synthase activity, b) transforming the polynucleotide into a host cell, c) cultivating the host cell to produce the polypeptide having EDDS synthase activity, and optionally d) purifying the polypeptide having EDDS synthase activity.
16. An isolated, synthetic, or recombinant polynucleotide encoding the isolated, synthetic, or recombinant polypeptide of any of preferred embodiments 10 to 14, wherein the isolated, synthetic, or recombinant polynucleotide preferably has with increasing preference at least 80%, at least 85%, at least 90%, at least 95%, at least 96%, at least 97%, at least 98%, at least 99%, or 100% sequence identity to SEQ ID NO: 2, SEQ ID NO: 8, or SEQ ID NO: 10, preferably, wherein when the polypeptide having EDDS synthase activity has at least 90% sequence identity to SEQ ID NO: 1, the polynucleotide encoding the polypeptide having EDDS synthase activity has at least 70% sequence identity to SEQ ID NO: 2, or wherein, when the polypeptide having EDDS synthase activity has at least 90% sequence identity to SEQ ID NO: 7, the polynucleotide encoding the polypeptide having EDDS synthase activity has at least 70% sequence identity to SEQ ID NO: 8, or wherein, when the polypeptide having EDDS synthase activity has at least 90% sequence identity to SEQ ID NO: 9, the polynucleotide encoding the polypeptide having EDDS synthase activity has at least 70% sequence identity to SEQ ID NO: 10.
17. A nucleic acid construct comprising the polynucleotide of embodiment 16.
18. An expression vector comprising the polynucleotide of preferred embodiment 16 or the nucleic acid construct of preferred embodiment 17.
19. A host cell comprising the polynucleotide of preferred embodiment 16, the nucleic acid construct of preferred embodiment 17, or the expression vector of preferred embodiment 18.
20. The host cell according to preferred embodiment 19, wherein the host cell is an E. coli cell.
21. The host cell according to any of preferred embodiment 19 or 20, wherein the host cell has a reduced or deleted fumarase activity.
22. A method for producing EDDS, wherein the method comprises the steps of
   a) providing a host cell according to any of the preferred embodiments 19 to 21;
   b) cultivating the host cell of step (a) under conditions conductive for the expression of polynucleotide encoding the EDDS synthase;
   c) optionally harvesting the host cell step (b); and
   d) contacting the host cell of step (b) and/or (c) with fumaric acid and ethylenediamine under conditions allowing the fumaric acid and ethylenediamine to be in contact with the EDDS synthase to produce EDDS, preferably under conditions as described in any of preferred embodiments 1 to 9.
23. A method of producing a composition, preferably a detergent composition, comprising EDDS, wherein the method comprises the steps of:
   a) producing EDDS using the method according to any of preferred embodiments 1 to 9, and
   b) formulating the EDDS produced under a) into the composition.
24. The method of producing a composition according to embodiment 23, wherein the composition comprising EDDS is a detergent composition and wherein the EDDS is in the form of the magnesium salt of EDDS.
25. The method of producing a composition according to embodiment 23 or 24, wherein the optical purity of the EDDS has with increasing preference of at least 90%, at least 91%, at least 92%, at least 93%, at least 94% at least 95%, at least 96%, at least 97%, at least 98%, or at least 99% enantiomeric excess (ee) of (S,S)-EDDS or is 100% (S,S)-EDDS.

### EXAMPLES

### Example 1 Cloning of EDDS Synthases

DNA sequences encoding potential EDDS synthases and EDDS06 as control were generated using standard codon usage of Escherichia coli. The DNA sequences were synthesized (BioCat GmbH) and cloned into the plasmid pDHE19.2 (Ress-Loeschke, M. et al., DE 19848129, 1998, (BASF AG)). The gene of interest lies under control of a rhamnose inducible promoter (rhaBAD). The resulting plasmids were used to transform competent cells (Chung, C. T. et al., Proc Natl Acad Sci USA, 1989, 86, 2172) of the E. coli strain TG10 (Kesseler, M. et al., WO2004050877A1, 2004, (BASF AG)). The E. coli strain TG10 is a rhaA⁻-derivate of E. coli TG1 (DSMZ 6056) transformed with pHSG575 (Takeshita, S. et al., Gene, 1987, 61, 63) and pAgro4 (pBB541 in Tomoyasu, T. et al., Mol. Microbiol., 2001, 40, 397).

TABLE 1
EDDS synthase sequences

| Name | SEQ ID NO polypeptide | SEQ ID NO polynucleotide |
|---|---|---|
| EDDS01 | 1 | 2 |
| EDDS02 | 3 | 4 |
| EDDS03 | 5 | 6 |
| EDDS04 | 7 | 8 |
| EDDS05 | 9 | 10 |
| EDDS06 (control) | 11 | 12 |

### Example 2 Recombinant Production of EDDS Synthases

E. coli TG10 carrying the recombinant plasmids of the potential EDDS synthases and the control were used to inoculate 20 ml LB medium (Bertani, G., J Bacteriol, 1951, 62, 293) supplemented with 100 μg/ml ampicillin, 50 μg/ml spectinomycin, 20 μg/ml chloramphenicol, 12.5 μg/ml tetracycline, 0.2 mM isopropyl-ß-D-thiogalactopyranosid, and 0.5 g/l rhamnose in a 100 ml baffled Erlenmeyer flask. The cultures were incubated at 37° C. for 18 h under shaking conditions (200 rpm).

Samples of whole cells were normalized to OD15 (600 nm) and analyzed using SDS PAGE. Band intensities of EDDS synthases were compared, and as a measure of expression level a signal factor was calculated, see Table 2. Therefore, the EDDS01 synthase signal was set to 1 and correlation factors (signal factors) were calculated in comparison to EDDS01.

TABLE 2
Expression level (signal factors) of EDDS
synthases determined using SDS PAGE

| | Signal factor (corresponding |
|---|---|
| | to band intensity in |
| EDDS synthase | SDS PAGE) |
| EDDS01 (SEQ ID NO: 1) | 1 |
| EDDS02 (SEQ ID NO: 3) | 0.54 |
| EDDS03 (SEQ ID NO: 5) | 0.73 |
| EDDS04 (SEQ ID NO: 7) | 0.48 |
| EDDS05 (SEQ ID NO: 9) | 0.35 |
| EDDS06 (SEQ ID NO: 11) (control) | 0.64 |

### Example 3 Activity Determination of EDDS Synthases

Enzymatic activity was measured at 37° C. by incubating 20 μl thawed cells expressing EDDS synthase (which were previously frozen at −20° C. in the cultivation medium), normalized to OD 15, in 100 μl substrate buffer containing 71.6 g/l fumaric acid, 26.95 g/l magnesium hydroxide and 18.5 g/l ethylenediamine in 1 M Tris buffer at pH 8.5. 10 μl samples were taken 30 mins after starting the reaction. The reaction was stopped by adding 290 μl stop/eluent solution (500 ml of 25% methanol, 1500 g water, 1 g/L copper II acetate-monohydrate, 2 g/L tetrabutylammoniumhydrate, pH 2.8 (adjusted with H3PO4)). Samples were then filtered through a 0.22 μm filter before being analyzed using HPLC analysis. For HPLC measurements the following settings were used: column: Aqua C18 (Phenomenex), temperature: 40° C., UV detection at 254 nm, isocratic run with eluent solution.

The volumetric enzymatic activity was calculated using the following formula:

$$volumetric activity [ \frac{k U}{L} ] = \frac{Δc [ \frac{mmol}{L} ]}{Δ t [ min ]} \cdot k$$

with delta C being the EDDS concentration produced in the time span delta t (here 0 to 30 min) and k as the dilution factor, which reflects the sample dilution.

To calculate the specific activities, the volumetric activities were divided by the respective signal factor (Table 3).

TABLE 3
Activities of EDDS synthases

| | Volumetric | Signal | Specific activity |
|---|---|---|---|
| | activity | factors | (kU/L*signal factor) |
| EDDS01 (SEQ ID NO: 1) | 3.43 | 1.00 | 3.43 |
| EDDS02 (SEQ ID NO: 3) | 0.00 | 0.54 | 0.00 |
| EDDS03 (SEQ ID NO: 5) | 0.00 | 0.73 | 0.00 |
| EDDS04 (SEQ ID NO: 7) | 0.97 | 0.48 | 2.01 |
| EDDS05 (SEQ ID NO: 9) | 0.02 | 0.35 | 0.05 |
| EDDS06 (SEQ ID NO: 11) | 0.76 | 0.64 | 1.18 |
| (control) | | | |

As can be derived from Table 3, EDDS01 (SEQ ID NO: 1), EDDS04 (SEQ ID NO: 7), and EDDS05 (SEQ ID NO: 9) show EDDS synthase activity. Surprisingly, EDDS02 (SEQ ID NO: 3) and EDDS03 (SEQ ID NO: 5) did not show any EDDS synthase activity. EDDS01 (SEQ ID NO: 1) and EDDS04 (SEQ ID NO: 7) showed particularly high EDDS synthase activity.

1. A method for producing EDDS using a polypeptide having ethylenediamine-N,N′-disuccinic acid (EDDS) synthase activity comprising contacting fumaric acid and ethylenediamine with an EDDS synthase under conditions allowing the EDDS synthase to produce EDDS, wherein the EDDS synthase comprises an amino acid sequence that is at least 87% identical to SEQ ID NO: 1, an amino acid sequence that is at least 80% identical to SEQ ID NO: 7, or an amino acid sequence that is at least 80% identical to SEQ ID NO: 9.

2. An isolated, synthetic, or recombinant polypeptide having EDDS synthase activity comprising an amino acid sequence that is at least 87% identical to SEQ ID NO: 1, an amino acid sequence that is at least 80% identical to SEQ ID NO: 7, or an amino acid sequence that is at least 80% identical to SEQ ID NO: 9.

3. The isolated, synthetic, or recombinant polypeptide according to claim 2, wherein the polypeptide comprises an amino acid sequence that is at least 87%, at least 90%, at least 92%, at least 95%, at least 97%, or 100% identical to SEQ ID NO: 1.

4. The isolated, synthetic, or recombinant polypeptide according to claim 2, wherein the polypeptide comprises an amino acid sequence that is at least 80%, at least 82%, at least 85%, at least 87%, at least 90%, at least 92%, at least 95%, at least 97%, or 100% identical to SEQ ID NO: 7.

5. The isolated, synthetic, or recombinant polypeptide according to claim 2, wherein the polypeptide comprises an amino acid sequence that is at least 80%, at least 82%, at least 85%, at least 87%, at least 90%, at least 92%, at least 95%, at least 97%, or 100% identical to SEQ ID NO: 9.

6. The isolated, synthetic, or recombinant polypeptide according to claim 2, wherein the polypeptide comprises or consists of SEQ ID NO: 1, SEQ ID NO: 7, or SEQ ID NO: 9, or wherein the amino acid sequence of the polypeptide having EDDS synthase activity comprises or consists of SEQ ID NO: 1, SEQ ID NO: 7, or SEQ ID NO: 9 with 1-20 amino acid substitutions.

7. An isolated, synthetic, or recombinant polynucleotide encoding the isolated, synthetic, or recombinant polypeptide according to claim 2, wherein the isolated, synthetic, or recombinant polynucleotide has at least 80%, at least 85%, at least 90%, at least 95%, at least 96%, at least 97%, at least 98%, at least 99%, or 100% sequence identity to SEQ ID NO: 2, SEQ ID NO: 8, or SEQ ID NO: 10.

8. A nucleic acid construct comprising the polynucleotide of claim 7.

9. An expression vector comprising the polynucleotide of claim 7.

10. A host cell comprising the polynucleotide of claim 7.

11. The host cell according to claim 10, wherein the host cell is an E. coli cell.

12. The host cell according to claim 10, wherein the host cell has a reduced or deleted fumarase activity.

13. A method of making an isolated, synthetic, or recombinant polypeptide, comprising the steps of a) providing a host cell of claim 10, b) cultivating the host cell to produce the polypeptide having EDDS synthase activity, and optionally c) purifying the polypeptide having EDDS synthase activity.

14. A method of producing a composition comprising EDDS, wherein the method comprises the steps of:
a) producing EDDS using the method according to claim 1, and
b) formulating the EDDS produced under a) into a composition.

15. An expression vector comprising the nucleic acid construct of claim 8.

16. A host cell comprising the nucleic acid construct of claim 8.

17. A host cell comprising the expression vector of claim 9.

18. The method of claim 14, wherein the composition is a detergent composition.
